(19) United States
(12) Patent Application Publication
(10) Pub. No.: US 2017/0150992 A1
(43) Pub. Date: Jun. 1, 2017

(54) Prosthetic Artificial Insemination Device

(71) Applicant: John-Klaus Powell, Boca Raton, FL (US)

(72) Inventor: John-Klaus Powell, Boca Raton, FL (US)

(21) Appl. No.: 14/953,368

(22) Filed: Nov. 29, 2015

(51) Int. Cl. A61B 17/43; A61H 23/00; A61H 19/00

(52) U.S. Cl. CPC A61B 17/43; A61H 19/50; A61H 19/40; A61H 19/44; A61H 23/00; A61H 2201/0103; A61H 2201/163

(57) ABSTRACT

A Biomimetic implant is an electromechanical implant which mimics the original function of the replaced body part very closely, or surpasses the functions of the replaced body part. The present invention will allow the wearer to artificially inseminate a sexual partner, experience an orgasm as a direct consequence of stroking the shaft of the invention, role play as a male, and serve as a non-surgical penile implant incorporating both aesthetic realism and functionality. Until now, no other dildo or prosthetic has ever existed with the full range of abilities of the invention presented here in. The ability to provide tactile feedback by use of sensors, pass urine, transition from flaccid to erect and also ejaculate make-up four of the most distinct features of the patent presented here in.

### CROSS-REFERENCE TO RELATED APPLICATIONS

This application claims benefit of priority under 35 U.S.C 119 to U.S. Provisional application Ser. No. 62/085,364, filed Nov. 28, 2014 and entitled “Prosthetic Artificial Insemination Device,” the contents of which are herein incorporated by reference in its entirety.

### BACKGROUND OF THE INVENTION

The field of the invention pertains to Bionic Implant, Dildo, Prosthetic Penis, Adult Toy, Novelty, Strapless Strap-on, Strap-on.

U.S. Pat. No. 0,224,482, is on a sexual device to be worn externally with the aid of a harness device by a woman, man, or transgender person. U.S. Pat. No. 0,224,482, allows said wearer to artificially inseminate a sexual partner by means of a syringe. The present said invention is set apart from such prior art by its unique mechanism of ejaculating and insemination.

U.S. Pat. No. 7,931,605, on a sexual device to be worn by a woman either for auto-erotic or during intercourse provides simultaneous stimulation to the G-spot and the clitoris of said wearer but is not designed to have the distinct appearance of the penis nor does it permit said wearer to penetrate the vagina or anus of a sexual partner while being worn by said user. The present invention proposed here in relates to a nonsurgical implant used for sexual stimulation especially by lesbians, and female to male transgender allowing females to experience sexual stimulation from a male's perspective when employed during auto-erotic stimulation, fellatio or intercourse. The present invention mentioned here in allows said user to penetrate the vagina or anus of a sexual partner while receiving tactile feedback directly related to the moment of the act.

U.S. Pat. No. 6,350,230, on a sexual aid device for sexual stimulation having an elongated tube member, has a compressible liquid containment unit on a lower end thereof. The compression means is configured to compress the liquid containment unit to thereby ejaculate liquid from the tube. Where as in the present invention proposed here in, the ejaculation fluid is located within the internal bulb assembly where the fluid may be warmed by natural body heat and ejaculation is achieved by means of a trigger pump which is activated during the stroking of the shaft of the invention.

U.S. Pat. No. 5,690,603, on an erogenic stimulator describes a two-sided dildo that is anchored in either the vagina or anus by the contraction forces of the muscles of the vagina or anus respectably. This patent and it's related design Pat. Nos. 5,853,362, and 0,228,218, do not transition from a flaccid state to an erect state, pass urine, provide tactile feedback to said user by means of a sensor and microcontroller, or ejaculate. Transition from flaccid to erect, the ability to pass urine, provide tactile feedback through the use of sensors and a microcontroller while also having the ability to ejaculate make-up four of the most distinct features of the patent presented here in.

U.S. Pat. No. 0,136,205, on an implement used for sexual stimulation, especially by women is a phallus body where the non-newtonian fluid itself acts as a control means for the resistance to deformation of the dildo, clearly consists of a modifiable rigidity which accurately simulate the situation of the penis during sex which changes degree of erection and therefore rigidity depending on the moment of the act. The before mentioned U.S. Pat. No. 0,136,205, fails to take into account the importance of the situation of the penis during ejaculation, and post ejaculation. Post ejaculation there is a distinct deformation of the penis which must be duplicated in order to adhere to the bionic principles of the proposed invention here in. In addition, the added stimulation gained through the physical response to ejaculating observed by the receiving partner is lost in U.S. Pat. No. 0,136,205, but gained in the present patent proposed here in. Furthermore U.S. Pat. No. 0,136,205, unlike the patent proposed here in fails to provide the added benefit of artificial insemination.

US patent No. 20050234292, on a Hydraulically-driven vibrating massager for sexual stimulation achieving vibrational stimulation through rhythmic deformation of the tubes within the massager and erection by means of a hydraulic actuator, acknowledges that the male penis becomes erect and remains as such by hydraulic pressure, and hence the consideration of the hydraulic characteristics of the penis as a sort of prior art. Pat. No. 20050234292, is designed for auto-erotic stimulation and not simultaneous stimulation of two sexual partners. Pat. No. 20050234292, fails to meet the need to simulate having a penis, does not become erect as a result of the mechanical action of stroking the shaft, does not pass urine and can not be warn without the aid of straps and therefore differs from the patent proposed here in.

U.S. Pat. No. 5,842,970, is on an external penile prosthetic adapted to be worn incorporating the penis in an effort to alleviate impotence. U.S. Pat. No. 5,842,970 is not capable of transitioning from flaccid to erect and does nothing to stimulate the wearer's clitoris or G-spot, if the wearer happens to be a female. Where as the present invention proposed here in does not incorporate the penis and is not intended to alleviate impotency. The present invention proposed here in, is different from all prior art and modes of facilitating female-to-male transformation, sexual stimulation and auto-erotic stimulation in that it is capable of transforming from a flaccid state to an erect state by means of hydraulic pressure electrically driven by a microcontroller responding to the sensor signal generated from the mechanical action of stroking the shaft. The present invention proposed here in, utilizes a noninvasive external catheter to facilitates the passing of urine and it is designed to ejaculate semen and/or other fluids. The present invention's design consist of a reservoir which contains either semen or other fluids, uses electronics to facilitate an erection, is rechargeable, and uses sensors to coordinate programed responses to specific mechanical action observed during an erotic act. Such mechanical acts relate to but are not limited to; stroking the shaft in a back and forth motion, fellatio, and penetration.

The present invention relates to a bionic penis used for a wide range of sexual stimulation especially by women for female-to-female intercourse, and female-to-male transgenders while also facilitating varying degrees of gender play for any number of possible configurations of sexual partners. The present invention formed substantially of an elastomeric material to produce a realistic, natural look and feel is designed to remain in a flaccid state prior to sexual use and therefore can be worn as a strapless “packer” phallus to facilitate a more complete female-to-male transgender transformation. In order to facilitate an erection the shaft of the invention must be stimulated by vigorously stroking the shaft back and forth. The stroking action activates a shake sensor and circuit which in-turn powers the electric pump which supplies fluid into the extension tube(s) causing expansion and erection. Continuous stroking of the shaft is necessary to maintain an erection. Without continuous stroking of the shaft the present invention eventually looses pressure and returns to the flaccid state. Within the urinary canal there exist an air differential pressure sensor for the purpose of activating the G-spot stimulator during the act of fellatio or penetration. Ejaculation is achieved by pressing a trigger located at the base of the shaft. The invention is designed in such away that the trigger may be depressed during the stroking action, as this method allows for a realistic and natural appearance of a male ejaculation. Stroking of the shaft also activates an optional clitoral vibrator as an added form of stimulation. The present invention is designed for both sexual and non-sexual use. When used as a packer phallus the present invention permits the user to urinate while standing without removal of the invention.

The invention is made strapless by the use of an attachment flange and an internal bulb assembly. The attachment flange is a strongly re-enforced polymer which may be adhered to the pubic area of the wearer. The insertion bulb serves three functions. The primary function of the insertion bulb is as a housing for the ejaculation fluid reservoir. The secondary function of the insertion bulb is as an anchor to keep the invention properly positioned. The third function of the insertion bulb is providing stimulation to the G-spot. Attached to the body of the insertion bulb is a component 10 the internal vibrator which is triggered on/off by component 15 the air pressure differential sensor located within component 1 the vulva cover. It will be understood that the particular material, method and device embodying the invention are shown by way of illustration and not as limitation of the invention. The principles and features of this invention may be employed in various and numerous embodiments without departing from the scope of the invention.

### BRIEF SUMMARY OF THE INVENTION

Bionics is the application of biological methods and systems found in nature to the design of engineering systems. In medicine, bionics means the replacement or enhancement of organs or other body parts by mechanical versions. A biomimetic implant is an electromechanical implant which mimics the original function of the replaced body part very closely, or surpasses the functions of the replaced body part. The present invention will allow the wearer to artificially inseminate a sexual partner, experience an orgasm as a direct consequence of stroking the shaft of the invention, role play as a male, and serve as a non-surgical penile implant incorporating both esthetic realism and functionality.

Currently there are several variations of dildos. There are numerous shapes, textures, and sizes of dildos. Some with straps and some that are strapless strap-ons. Some dildos are soft, some are flexible, some are rigid and some are bent to visually appear limp. There are very few that can go from limp to erect. None that incorporate the full range of abilities of the invention presented here in.

Amongst the prosthesis that do transform from a soft to a rigid state, none become erect by means of vigorously stroking the shaft back and forth, utilizing the signal generated through the mechanical action of stroking the shaft to facilitate an erection. Further more, those female urination devices (FUD) which serve as pack and play dildos do not ejaculate fluids and therefore they do not employ the same mechanisms of the invention here in. The ability to provide tactile feedback by use of sensors, pass urine, transition from flaccid to erect and also ejaculate make-up four of the most distinct features of the patent presented here in.

### BRIEF DESCRIPTION OF DRAWING

For further understanding of the nature and objects of the present invention, reference should be made to the following description and taken in conjunction with the accompanying drawings in which like parts are given like references numerals wherein:

FIG. 01 is a perspective view of the internal components of the present invention represented in an erect state.

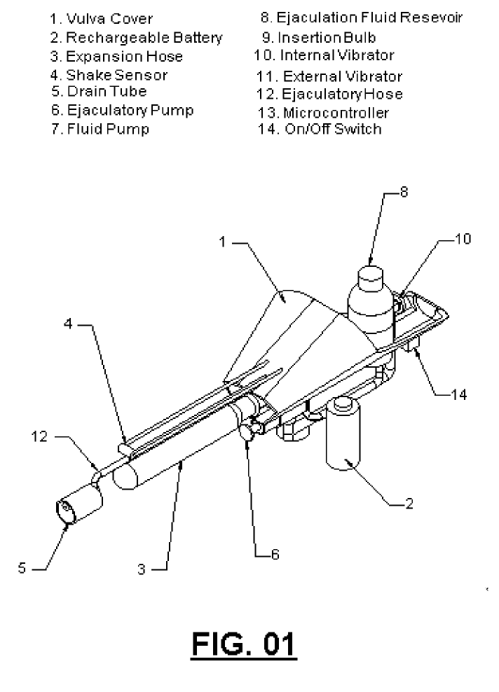

FIG. 02 Depicted here is an illustration of one possible iteration of the present invention with the internal components enveloped in component 16 an elastomeric material, such as, but not limited to silicone, latex. or polyvinyl.

FIG. 03 is a top view of the internal components of the present invention. It is from this view that components 11 is visible. FIG. 03 depicts component 11, a twin set of vibrators, positioned to stimulate the clitoris on the left and right sides. Component 11 maybe physically repositioned by the user so that it may better align with the specific anatomy of said user.

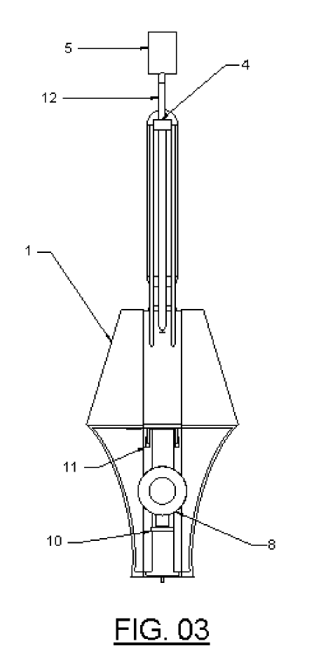

FIG. 04 is a side view of the internal components of the present invention. FIG. 04 depicts components 1, 2, 3, 4, 5, 6, 7, 8, 10, 11, 12 and 14. Where component 1 is the Vulva cover, a stretchy and flexible oval shaped flap connecting to the shaft and fluid pump. Component 1 attaches to the pubic area, covers the vulva and provides a flush and seamless connection of the invention with the body. Component 2 is a set of rechargeable batteries strategically positioned to simulate testicles. Component 4 is a shake sensor. Component 5 is the drain tube, a fixed dimension cylinder position for the purpose of allowing passage and draining of fluids. Component 6 is the ejaculation pump, compression pump used to expel fluid housed within component 8. Component 8 is a fluid reservoir having a hollow core used to house the ejaculation fluid and component 7 is an electric fluid pump used to draw fluid and pressurize component 3 the expansion hose. Component 10 is the internal vibrator positioned to stimulate the G-spot and other erogenous zones. Component 12 is the ejaculatory hose, a connecting hose which extends from component 6 to the tip of the shaft allowing for the expelling of fluids contained within component 8.

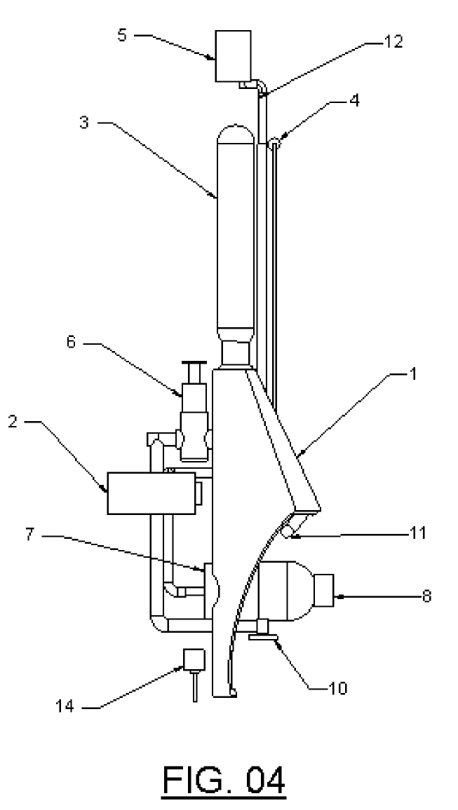

FIG. 05 is a rear view of the invention depicting components 1,2,7, 8, 10 and 14. Component 14 is an on/off switch, allowing for the activation of the invention presented herein.

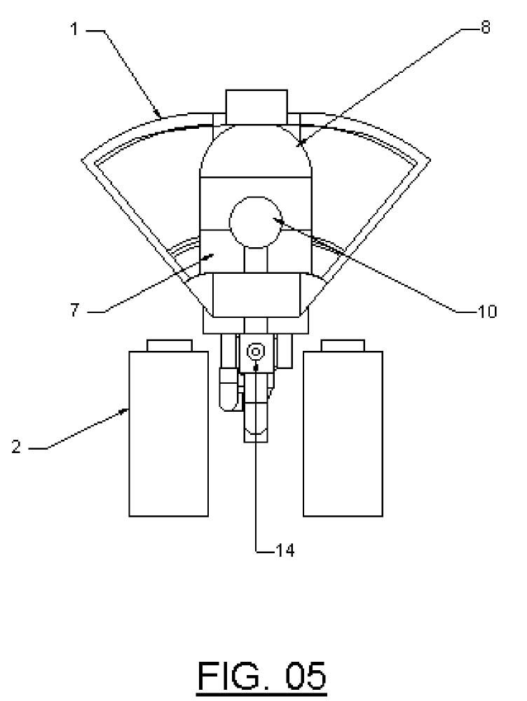

FIG. 06 is a front, view of the invention depicting components 1,2,4,5,6,8 and 12.

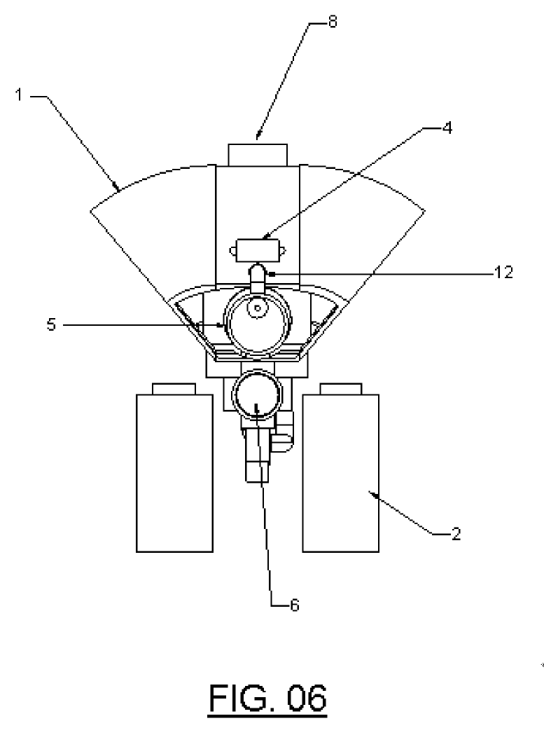

FIG. 07 is a bottom, view of the invention depicting components 1,2,3,5,6,7,12, 14 and 19. Where component 19 is opening 1 on component 7 the fluid pump. Component 19 draws fluid to permit the transfer of fluids through component 7 and out of component 20 otherwise referred to as opening 2 and into component 3 the expansion hose 3.

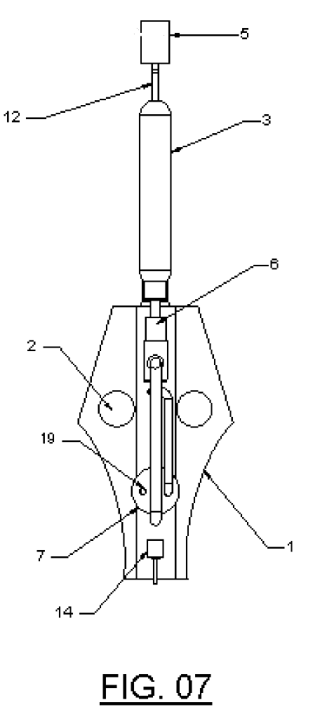

FIG. 08 is an isolated front view of component 8 and component 7. Depicted here is components 7 the fluid pump, component 8 the reservoir, component 19 and 20. Components 19 and 20 are opening 1 and opening 2 respectively.

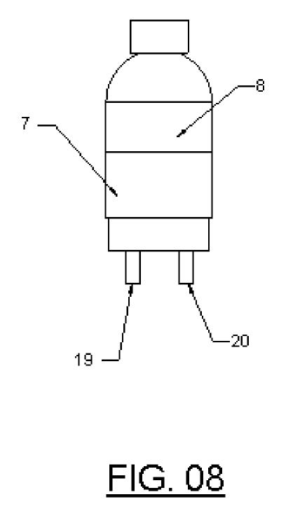

FIG. 09 is a top view of component 13, the microcontroller. The microcontroller is a programmable circuit board which monitors sensor readings and execute commands.

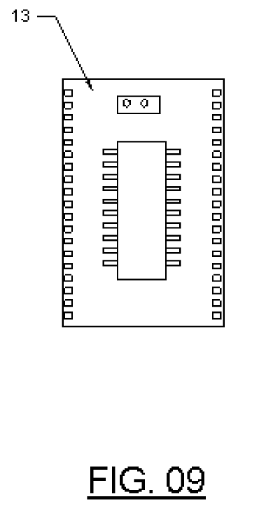

FIG. 10 is a side view of component 13. depicted here below component 13 is component 15, an air pressure differential sensor. This on/off switch responds to the change in air pressure exerted on to the invention during the act of sexual stimulation. The readings are analyzed by component 13 and the appropriate command is sent to either component 10, component 7 or both dependent on the moment of the act.

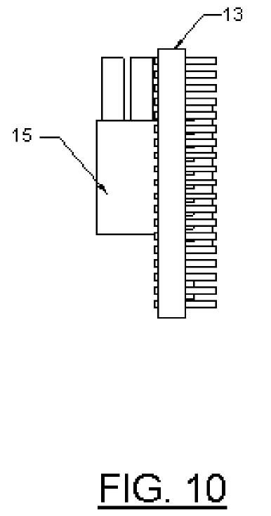

FIG. 11 Depicted here is an illustration of the internal components of the present invention alien with the wearer's body.

FIG. 12 Depicted here is an illustration of one possible iteration of the present invention shown in position attached to the body of the wearer.

FIG. 13 Depicted here is an illustration of a cross section view of one possible iteration of the present invention in use for penetration during auto-erotica. FIG. 13 depicts component 18 the crown and component 17 the shaft. Component 17 is a hollow and generally cylindrical or phallic like shaft comprised of an elastomeric material. Component 16 is an elastomeric cover housing components 1, 2, 3, 4, 5, 6, 7, 8, 10, 12, and 14 while serving as the reservoir for the fluid to be drawn by component 7 to fill component 3.

### DETAILED DESCRIPTION OF THE INVENTION

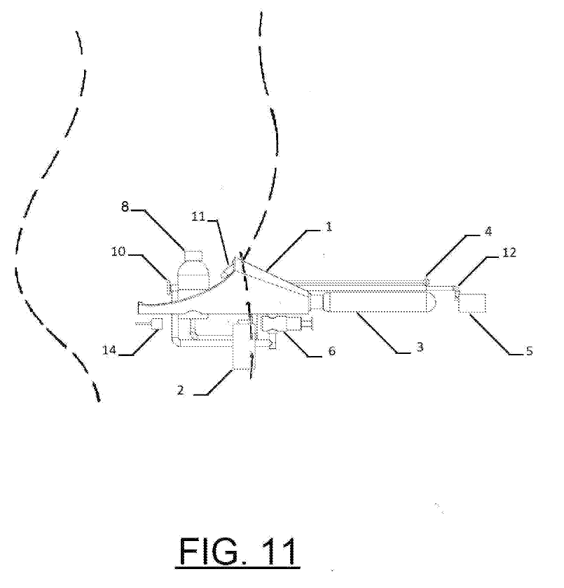

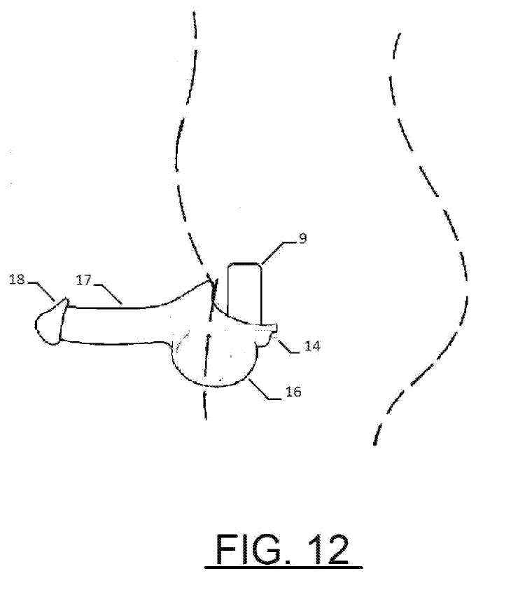

As a bionic prosthesis with tactile feedback used as a female urination device (FUD) and furthermore for a wide range of sexual stimulation especially by women, lesbians, and female to male transgenders with the added benefit of artificial insemination, while also facilitating various degrees of gender play. Shown in FIG. 11 and FIG. 12, as it would appear when attached to the body. It will also be noted that the invention proposed herein may also be fitted to a robotic body and there by providing a bionic sex organ for such devices as robotic sex dolls, synthetic humans and all other robotic and/or machine systems employing a phallus or phallic like part.

As shown, the bionic prosthesis is generally cylindrical or phallic like and having two ends with one end being the insertion bulb 9 and the other being a hollow shaft 17 having an expandable elastomeric expansion hose 3 centered therein the hollow core utilized for the transition of the invention from a flaccid state to an erect state. Hollow shaft 17, being configured as a phallic emulator or hose of typical design being generally cylindrical in shape with an enlarged tip or crown 18 as shown in FIG. 12. It will be noted, the actual design of expansion hose 3 and hollow shaft 17 may vary.

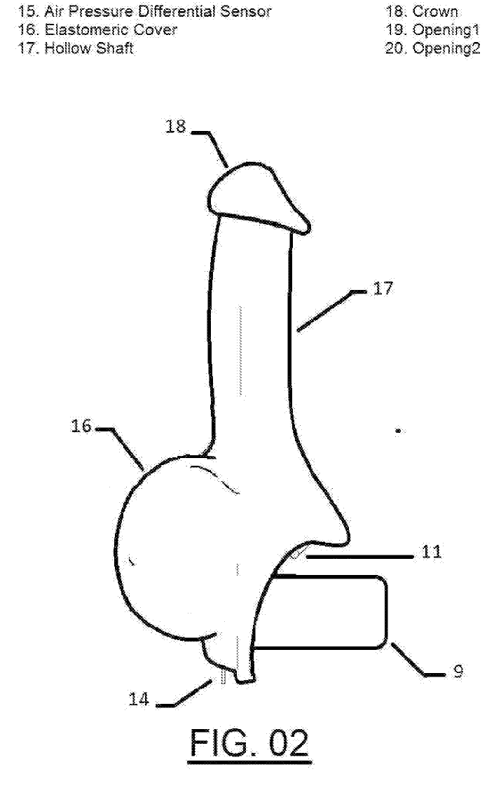

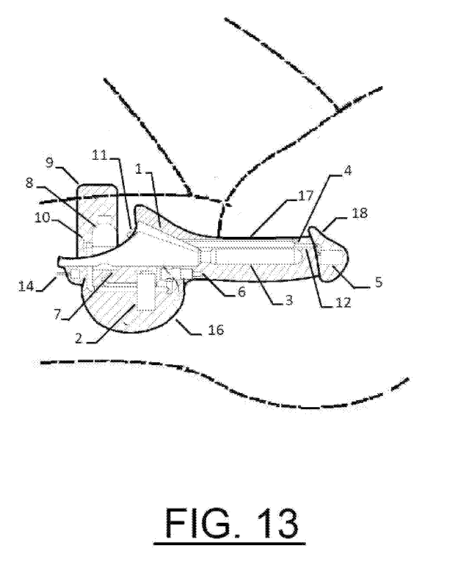

The insertion bulb 9 is connected or coupled to the enlarged saddle or vulva cover 1 as best shown in FIGS. 02, 12, and 13. The vulva cover 1 is generally integrally connected with hollow shaft 17 and elastomeric expansion hose 3 as shown in FIG. 02. The vibrator 10 and vibrator 11 are positioned to stimulate the G-spot and clitoris respectably. Vibrator 10 the G-spot vibrator is connected and controlled by the microcontroller 13 in such away that when the air pressure differential sensor 15 is activated by the particular moment of the act being either fellatio or penetration, vibrator 10 is switched on. Note that vibrator 10 is strictly dependent on the activation of sensor 15 and therefor can not be activated by strictly stroking the hollow shaft 17. The activation of vibrator 10 during the act of fellatio is independent of the activation of vibrator 11 and sensor 4 allowing for varying degrees of stimulation dependent on the technique of the partner or the moment of the act. The isolated activation of vibrator 10 demands the participation of a partner and in this way promotes the interaction between people. Vibrator 11 the clitoral vibrator is connected and controlled by the microcontroller 13 in such away that when the shake sensor 4 is activated by the particular moment of the act being either fellatio, penetration, or otherwise stroking of the shaft 17 vibrator 11 is switched on and use thereby providing additional means of stimulation for the wearer. Vibrator 11 may be removable or it may be a permanent part of said invention.

Bulb 9 provides a means of holding said invention in place during use, as best seen in FIG. 12, such as by inserting bulb 9 inside either the vaginal or anal cavity of the wearer. Bulb 9 serves two functions. One function of bulb 9 is to provide stimulation whether inserted vaginally or rectally. In either type of insertion, the bulb 9 provides a means for the vaginal or sphincter muscle groups to hold bulb 9 in place with additional support provided by reusable silicone adhesive positioned along the lining of the vulva cover 1. Thus, by simply inserting bulb 9 within the wearer, the invention proposed herein is retained in place by that wearer's muscle groups with additional support being provided by the adhesion to the body of said wearer by the silicone adhesive lining the vulva cover 1. A secondary function of Bulb 9 is as the housing unit for reservoir 8, the removable and refillable container used to hold such fluids as either semen or lubrication.

Consequently, phallic hollow shaft 17 may be utilized to stimulate the other partner while the insertion bulb 9 and vibrator 10 simultaneously stimulate the wearing partner should such wearing partner be female. However, should the wearing partner be male, vibrator 11 can be removed so as to not interfere with such use.

One unique feature of the invention proposed herein is its ability to vibrate in response to the stroking of the shaft 17 and sucking of the crown 18. So as to offer tactile feedback in correlation with the moment of the act. Furthermore, the ability of the invention proposed herein to transition from flaccid to erect without the use of a squeeze pump further sets apart the invention from all other prior art. The invention proposed herein transitions from a flaccid state to an erect state in response to either the stroking of shaft 17 or sucking of crown 18 by the use of a microcontroller 13, shake sensor 4, air pressure differential sensor 15 and micro fluid pump 7. The activation of sensor 4 or sensor 15 sends a signal to the microcontroller 13 to turn on the micro fluid pump 7 which in-turn fills expansion hose 3. the elimination of the need for any type of strapping device greatly enhances the use of the invention proposed herein, by eliminating any delay or discomfort normally associated with such straps.

The actual length and diameter of shaft 17 and bulb 9 can vary as needed depending on the needs of the partners. The particular variation of the size of shaft 17 is determined via the amount of fluid contained within the component 16. The design composition of component 16 the elastomeric cover consists of a fluid reservoir section having an outward appearance with the likeness of testicles. Component 19 and component 20 are openings found on component 7 the fluid pump, where component 19 is opening 1 and component 20 is opening 2. Opening 1 of pump 7 is connected to the inner layer of component 16 the elastomeric cover in such a way that permits the transfer of fluids out of component 16, into component 7 and through opening 2 to fill expansion hose 3. The process is then completed in the reverse order to permit the deflation of expansion hose 3 reverting shaft 17 back to a flaccid state.

The invention proposed herein is further distinguished from other prior art by its ability to function as a female urination device (FUD). This function is observed by the unique ability of the invention to pass urine utilizing the hollow shaft 17 as an external noninvasive catheter. Opening 1 of the hollow shaft 17 located at the front end of the vulva cover 1 below the detachable vibrator 11 allows for a natural flow of fluids through the hollow shaft 17 and out the distal end of crown 18. This particular feature is distinctively fitted for female to male transgenders giving acknowledgment to their particular need to utilize public restrooms while protecting their need to honor their gender expression. This fact does not in any way take away the freedom of any female to utilize the FUD functionality of the invention proposed herein but rather allows the freedom to explore or otherwise experiment with a wide range of gender play.

Artificial insemination is a key component of the invention proposed herein. This distinctive feature is observed by the activation of pump 6. Pump 6 is attached to reservoir 8 by means of an elastomeric tube allowing for the one way flow of the contained fluids within reservoir 8 (be it either lubricant or semen) along ejaculatory hose 12 and finally out the distal opening of crown 18. Pump 6 is strategically positioned so as to allow for the triggering of said ejaculation by means of stroking the hollow shaft 17 or during the act of penetration. Such positioning acknowledge the natural functioning of a biological penis and therefore adheres to the biomimetic nature of the invention proposed herein. The invention proposed herein acknowledge the need for a biological female to experience fatherhood in an as close to natural way as possible and consequently is engineered to provide pseudo real means of achieving said goal.

1. A strapless bionic prosthesis with tactile feedback and a hollow generally cylindrical or phallic like shaft utilized for the passing of urine.
(a) A strapless bionic prosthesis employing the use of a microprocessor, vibrator(s), air pressure differential sensor and longitudinal motion sensors.
(b) An artificial insemination device employing a pump mechanism having two ends. The first end being connected to a detachable reservoir by means of an elastomeric hose and the second end being connected to the opening of the hollow and generally cylindrical or phallic like shaft of the main body of said invention by means of a second elastomeric hose.
(c) A stand to pee device attaching to the wearer's body by means of an insertion bulb, comprising an elastomeric body enclosing an electric fluid pump, and detachable refillable reservoir.
(d) A stand to pee device having an elastomeric hose housed within the hollow and generally cylindrical or phallic like shaft of the main body of said invention with connection to a fluid pump for pressurizing and depressurizing of said elastomeric hose.

2. A strapless bionic prosthesis as proposed in claim 1, having an elastomeric hose housed within the shaft of said invention for the purpose of providing both radial and longitudinal expansion of said shaft and there by achieving a dynamic physical state change and appearance of a flaccid to erect penis.

3. As proposed in claim 1, A strapless bionic prosthesis having the functionality of transitioning from a generally flaccid state to a full erect state with automatic return to the flaccid state.

4. A strapless bionic prosthesis having G-spot and clitoral stimulation for the wearer during intercourse with a partner.

5. A strapless bionic prosthesis G-spot and clitoral stimulation for the wearer while simulating penile stimulation.

6. A strapless bionic prosthesis G-spot and clitoral stimulation for the wearer while the partner uses the invention to perform fellatio.

7. As proposed in claim 1, a strapless bionic prosthesis have a pump mechanism having two ends for ejaculation of a house fluid

8. As proposed in claim 7, a strapless bionic prosthesis having a system and mechanism for employing Artificial Insemination

9. As proposed in claim 1, a strapless bionic prosthesis having a hollow and generally cylindrical or phallic like shaft for the passing of urine while attached to the body of the wearer.

10. A strapless bionic prosthesis having vibrator(s) with adjustable positioning.
